(19) United States
(12) Patent Application Publication
(10) Pub. No.: US 2026/0041926 A1
(43) Pub. Date: Feb. 12, 2026

(54) FLEXIBLE THREE-DIMENSIONAL ELECTRONIC DEVICE FOR BIO-CONFORMAL ATTACHMENT

(71) Applicant: KOREA ADVANCED INSTITUTE OF SCIENCE AND TECHNOLOGY, Daejeon (KR)

(72) Inventors: Keon Jae LEE, Daejeon (KR); Jaehee LEE, Daejeon (KR); Minseo KIM, Daejeon (KR); Ki Yun NAM, Daejeon (KR)

(73) Assignee: KOREA ADVANCED INSTITUTE OF SCIENCE AND TECHNOLOGY, Daejeon (KR)

(21) Appl. No.: 19/289,897

(22) Filed: Aug. 4, 2025

(30) Foreign Application Priority Data
Aug. 6, 2024 (KR) 10-2024-0104982
Apr. 21, 2025 (KR) 10-2025-0051705

(51) Int. Cl. A61N 5/06

(52) U.S. Cl. CPC A61N 5/0601; A61N 2005/0632

(57) ABSTRACT

A flexible three-dimensional electronic device for bio-conformal attachment is provided. The flexible three-dimensional electronic device includes a central support having a predetermined area, a plurality of flexible supports connected to the central support and radially extending in multiple directions; and a plurality of light sources respectively attached to the plurality of flexible supports.

This application claims priority to and benefits of Korean Patent Application Nos. 10-2024-0104982 and 10-2025-0051705, under 35 U.S.C § 119, filed on Aug. 6, 2024, and Apr. 21, 2025, respectively, in the Korean Intellectual Property Office, the contents of which are incorporated herein in its entirety by reference.

### BACKGROUND

### 1. Technical Field

The present disclosure relates to a flexible three-dimensional electronic device for bio-conformal attachment, and more particularly, to a flexible three-dimensional electronic device for bio-conformal attachment that enables complete and damage-free adhesion to an organ without the use of adhesives and also allows for safe removal.

### 2. Description of the Related Art

Electronic devices for treating diseases have recently gained attention as medical devices owing to their ability to provide continuous treatment with minimal side effects. However, conventional electronic devices for treating diseases typically employ a two-dimensional (2D) planar structure, making it difficult to achieve sufficient adhesion to three-dimensional organs.

Despite attempts to improve adhesion by employing biocompatible adhesives, these approaches often lead to adverse effects, such as inflammation, due to the adhesive penetrating into the organ tissue over prolonged use. Furthermore, given the varying sizes and shapes of internal organs, treatments relying on a single structural form are inherently limited.

Currently available electronic devices for disease treatment are generally categorized into planar-type electronic devices and implantable-type electronic devices. Planar-type devices adhere to a surface area corresponding to the entire target organ but are prone to detachment under physical activity or impact. Moreover, when applied to curved surfaces, the planar-type devices are incompletely adhered, thereby reducing therapeutic effectiveness. On the other hand, implantable-type devices address detachment issues due to proximity by being directly inserted into the target organ, but the mismatch in Young's modulus between the target organ and the implanted device often causes tissue damage and inflammation.

### SUMMARY

The present disclosure aims to provide a flexible three-dimensional electronic device for bio-conformal attachment that is capable of secure and complete adhesion to an entire organ without the use of adhesives and allows for removal without causing damage.

In an aspect of the disclosure, the flexible three-dimensional electronic device for bio-conformal attachment includes a plurality of light sources and a support on which the plurality of light sources are attached to one surface thereof. The support includes a plurality of incision portions, each incision portions widening outwardly from a center along the incision portions.

In an embodiment, the incision portions are symmetric about the center of the support.

The disclosure also provides a flexible three-dimensional electronic device for bio-conformal attachment that includes: a central support having a predetermined area; a plurality of flexible supports connected to the central support and radially extending in multiple directions; and a plurality of light sources attached, respectively, to the plurality of flexible supports.

In an embodiment, the plurality of flexible supports include bending portions.

In an embodiment, the flexible supports are configured to be bent autonomously in response to internal physiological conditions.

In an embodiment, the flexible supports are equipped with a flexible insertion member attached to the central support and configured to be inserted toward biological tissue to which the flexible three-dimensional electronic device is adhered.

In an embodiment, the flexible insertion member is a protruding structure formed by folding a portion of the flexible support.

In an embodiment, the flexible insertion member is provided with a plurality of light sources.

In an embodiment, the aspect ratio of the plurality of flexible supports is equal to or greater than 4.0.

The disclosure also provides a flexible three-dimensional electronic device for bio-conformal attachment that includes: a central support in a polygonal or circular shape having a predetermined area to which a plurality of light sources are attached; and a plurality of flexible supports connected with the central support and radially extending in multiple directions, wherein incision portions between the flexible supports are symmetric about the central support.

In an embodiment, the flexible supports include second flexible supports disposed between first flexible supports. The first flexible supports become wider toward their ends as they extend from the central support, while the second flexible supports become narrower toward their ends as they extend from the central support. Accordingly, the gap between each first flexible support and adjacent second flexible support is widest at the distal ends of the first flexible support and second flexible support.

In an embodiment, the first and second flexible supports are configured to meet at their ends when the flexible three-dimensional electronic device is attached to biological tissue and bends in one direction.

In an embodiment, a hemispherical shape is formed by allowing the first and second flexible supports to meet at their ends, thereby enabling the device to conform to curvatures of organs and tissues.

The flexible three-dimensional electronic device for bio-conformal attachment according to the present disclosure enables full adhesion to target organs of various sizes and shapes and facilitates the treatment of diverse internal organs through its structural adaptability. Furthermore, it reduces the burden of extended open surgeries, allowing for more efficient treatment processes. Owing to the flexibility and 3D conforming structure, the device ensures full adhesion to curved surfaces, enabling applications across various fields, such as photothermal therapy (PTT), photobiomodulation therapy (PBMT), and photodynamic therapy (PDT).

This work was supported by the National Research Foundation of Korea (NRF) grant funded by the Korea Government (MSIT) (RS-2024-00406240). This work was supported by the Industrial Technology Innovation Program (RS-2024-00432143) funded By the Ministry of Trade, Industry & Energy (MOTIE, Korea).

### BRIEF DESCRIPTION OF THE DRAWINGS

These and/or other features will become apparent and more readily appreciated from the following description of the embodiments, taken in conjunction with the accompanying drawings in which:

FIG. 1 is a plan view of a flexible three-dimensional electronic device for bio-conformal attachment according to an embodiment of the present disclosure;

FIG. 2 is a perspective view of the flexible three-dimensional electronic device for bio-conformal attachment according to an embodiment of the present disclosure, and FIG. 3 illustrates each layer of a flexible support;

FIG. 4 shows photographs of the flexible three-dimensional electronic device for bio-conformal attachment manufactured according to an embodiment of the present disclosure;

FIG. 5 shows photographs of the electronic device according to an embodiment of the present disclosure;

FIG. 6 illustrates experimental results for the flexible three-dimensional electronic device for bio-conformal attachment manufactured according to an embodiment of the present disclosure;

FIG. 7 shows results of analysis of the optical characteristics of a light source (micro-LED) before and after transformation from a two-dimensional configuration to a three-dimensional configuration;

FIG. 8 shows results of analysis of the electrical and optical characteristics of the flexible three-dimensional electronic device according to the present disclosure over a 14-day period after transformation from a two-dimensional configuration to a three-dimensional configuration, as well as after 1,000 cycles of structural deformation;

FIG. 9 shows results of analysis after immersing the flexible three-dimensional electronic device according to the present disclosure in a PBS solution for six days following transformation from a two-dimensional structure to a three-dimensional structure;

FIG. 10 is a three-dimensional schematic view of a flexible three-dimensional electronic device according to another embodiment of the present disclosure, and FIG. 11 is a step-by-step schematic view illustrating a method of manufacturing a three-dimensional electronic device equipped with an insertion member shown in FIG. 10;

FIG. 12 is a plan view of a flexible three-dimensional electronic device having a wavy bending portion according to another embodiment of the present disclosure;

FIG. 13 is a plan view of a flexible three-dimensional electronic device for bio-conformal attachment according to yet another embodiment of the present disclosure;

FIG. 14 illustrates how the flexible three-dimensional electronic device shown in FIG. 13 is used after attachment;

FIG. 15 is a plan view of a flexible three-dimensional electronic device according to yet another embodiment of the present disclosure;

FIG. 16 is a cross-sectional view illustrating the interlayer structure of a flexible support according to the present embodiment, along with a table showing physical property parameters for each material;

FIGS. 17 to 19 show, respectively, the initial state, deformed state, and displacement plot of a substrate forming the flexible support, tested using different aspect ratios of the substrate; and

FIG. 20 shows summarized results based on the data from FIGS. 17 to 19.

### DETAILED DESCRIPTION

References will now be made in detail to certain embodiments, of which examples are illustrated in the accompanying drawings, where like reference numerals refer to like elements throughout. The embodiments may have a variety of forms and permutations, but the present disclosure shall by no means be construed as being limited to the described embodiments. Rather, the present disclosure shall be construed to encompass all forms, permutations, equivalents and substitutes covered by the technical ideas and scope of the present disclosure. Accordingly, the embodiments are merely described below, by referring to the figures, to explain features of the present disclosure.

Before proceeding with a detailed description, it should be noted that the terminology and expressions used in this specification are not intended to be limited to conventional or dictionary-defined meanings. Rather, the terms and expressions are to be understood in the context of the concepts defined and utilized by the inventor to describe the disclosure in the most effective manner.

Moreover, these terms and expressions should be interpreted as meanings and concepts that are in line with the technical ideas and scope of the present disclosure. That is, the terms appearing in the present specification are used for the purpose of describing particular embodiments and are not intended to limit the disclosure. It shall be appreciated that these terms have been defined to encompass various possibilities within the scope of the present disclosure.

Furthermore, in the present specification, singular expressions shall be understood to include their plural counterparts unless dictated otherwise in the context. Similarly, plural expressions may encompass singular meanings when the context allows.

Throughout the specification, when an element is described as “including” another element, it does not necessarily mean that any other elements are precluded but may be further included, unless expressly stated otherwise. Furthermore, if an element is described as being “present inside” or “connected to” another element, it is intended to indicate that the element may be directly connected to or in contact with the other element.

The present disclosure addresses the aforementioned issues by providing a novel form of flexible three-dimensional electronic device configured to adhere securely to an entire organ without using adhesives and to enable damage-free removal. To this end, the disclosure employs a two-dimensional planar substrate that can be incised to conform closely to the non-planar surface of the organ, enabling comprehensive adhesion across the organ's surface.

To this end, the disclosure provides a flexible three-dimensional electronic device for bio-conformal attachment, which includes a plurality of light sources and a support on which the plurality of light sources are mounted on one surface thereof. The support includes a plurality of incision portions and becomes wider outwardly from a center along the incision portions. The incision portions are symmetrically arranged about the center of the support.

Hereinafter, the present disclosure will be described in further detail through certain preferred embodiments.

### Embodiment 1

FIG. 1 is a plan view of a flexible three-dimensional electronic device for bio-conformal attachment according to an embodiment of the present disclosure. Referring to FIG. 1, a flexible three-dimensional electronic device configured for secure and complete adhesion to an entire organ without the use of adhesives and for removal without damage includes a central support 100 having a predetermined area in a polygonal or circular shape, a plurality of flexible supports 110 connected to the central support 100 and radially extending in multiple directions, and a plurality of light sources L attached, respectively, to the plurality of flexible supports 110. Incision portions are formed between the flexible supports, and it can be observed that the incision portions become wider outwardly from a center of the device.

In the present embodiment, the central support and the flexible supports may include a flexible plastic, metal electrodes formed on the flexible plastic, and a control chip configured to control the light sources. The angles of the supports may be flexibly adjusted based on the characteristics of the flexible plastic.

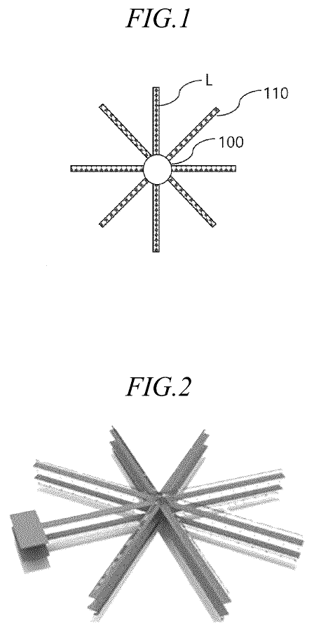

FIG. 2 is a perspective view of the flexible three-dimensional electronic device for bio-conformal attachment according to the present embodiment, and FIG. 3 illustrates each layer of the flexible support 100. Referring to FIGS. 2 and 3, the three-dimensional electronic device may include a transparent polyurethane (PU) substrate, holes formed in the PU substrate through which light from the light sources can be emitted, and serpentine-shaped electrodes formed over the holes.

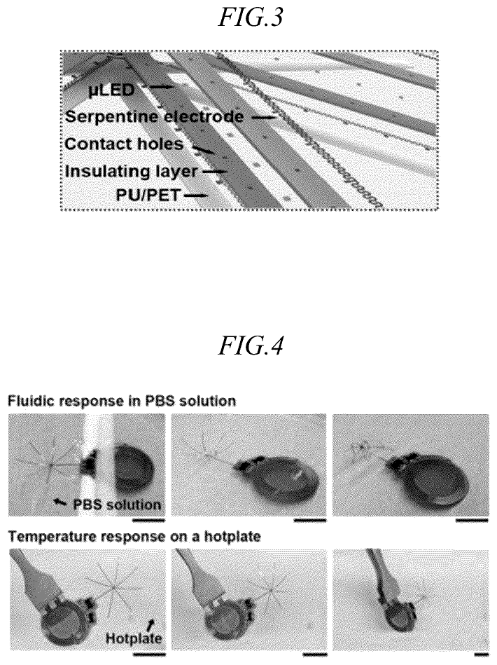

FIG. 4 shows photographs of the flexible three-dimensional electronic device manufactured according to an embodiment of the present disclosure. In particular, FIG. 4 shows the bending behavior of the electronic device in a phosphate-buffered saline (PBS) solution and under high-temperature conditions. Referring to FIG. 4, the flexible device exhibits self-folding behavior in response to external stimuli such as PBS solution (body fluids) and temperature (body heat), indicating that the device is expected to promote conformal adhesion to organs in a biological environment.

FIG. 5 shows photographs of the electronic device according to the embodiment.

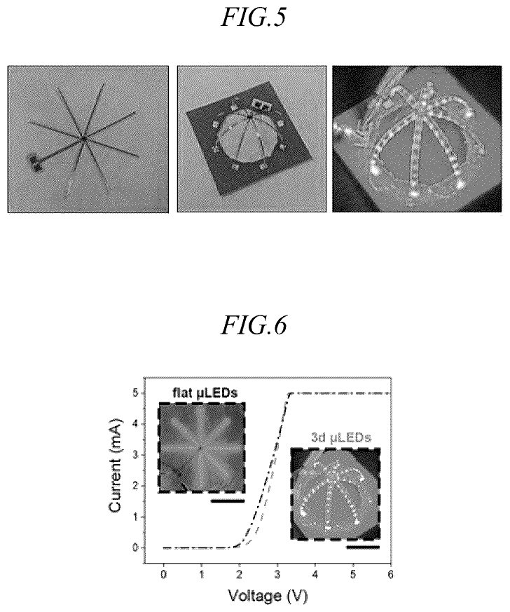

FIG. 6 illustrates experimental results for the flexible three-dimensional electronic device for bio-conformal attachment manufactured according to an embodiment of the present disclosure. Referring to FIG. 6, even after transforming the device from a two-dimensional configuration to a three-dimensional configuration, there is negligible difference in the forward voltage, which is an electrical characteristic of the electronic device shown in FIG. 5.

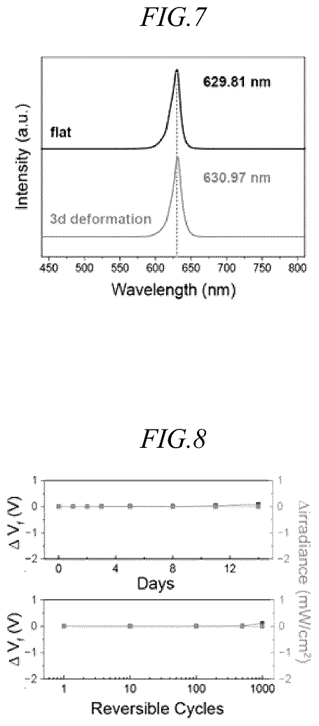

FIG. 7 shows results of analysis of the optical characteristics of a light source (micro-LED) before and after transformation from a two-dimensional configuration to a three-dimensional configuration. Referring to FIG. 7, it is evident that the peak wavelength remains nearly unchanged, which is consistent with the results shown in FIG. 6.

FIG. 8 shows results of analysis of the electrical and optical characteristics of the flexible three-dimensional electronic device according to the present disclosure over a 14-day period after transformation from a two-dimensional configuration to a three-dimensional configuration, as well as after 1,000 cycles of structural deformation. Referring to FIG. 8, it is observed that the device maintains stable electrical and optical characteristics even after 14 days and deformation of 1,000 times.

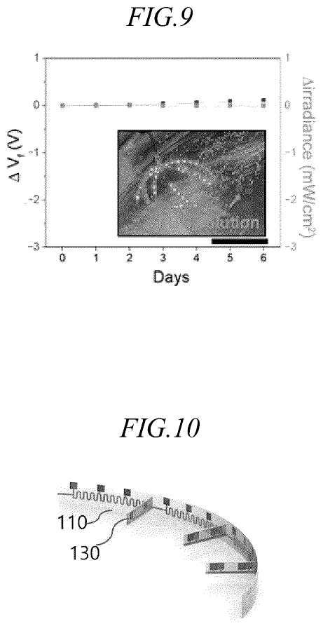

FIG. 9 shows results of analysis after immersing the electronic device according to the present disclosure in a PBS solution for six days following transformation from a two-dimensional configuration to a three-dimensional configuration. Referring to FIG. 9, the electrical and optical characteristics show little change even after the six days of immersion.

These results demonstrate that, compared to planar devices (flat μLEDs), the flexible three-dimensional electronic device (3D μLEDs) of the present disclosure provides superior adhesion to biological tissue and retains its performance even after deformation into a 3D configuration.

### Embodiment 2

FIG. 10 is a three-dimensional schematic view of a flexible three-dimensional electronic device according to another embodiment of the present disclosure, and FIG. 11 is a step-by-step schematic view illustrating a method for manufacturing a three-dimensional electronic device equipped with an insertion member shown in FIG. 10.

Referring to FIG. 10, in the present embodiment, the flexible support 110 is equipped with a flexible insertion member 130, which is attached to the flexible support 110 and configured to be inserted in the direction of biological tissue to which the flexible three-dimensional electronic device is to be adhered.

In the present embodiment, as illustrated in FIG. 11, the flexible insertion member 130 may be fabricated by folding the flexible support 110 and may be provided with a plurality of light sources. The boxes shown with broken red lines in FIG. 11 represent cross-sectional views of respective steps of the process for manufacturing the device.

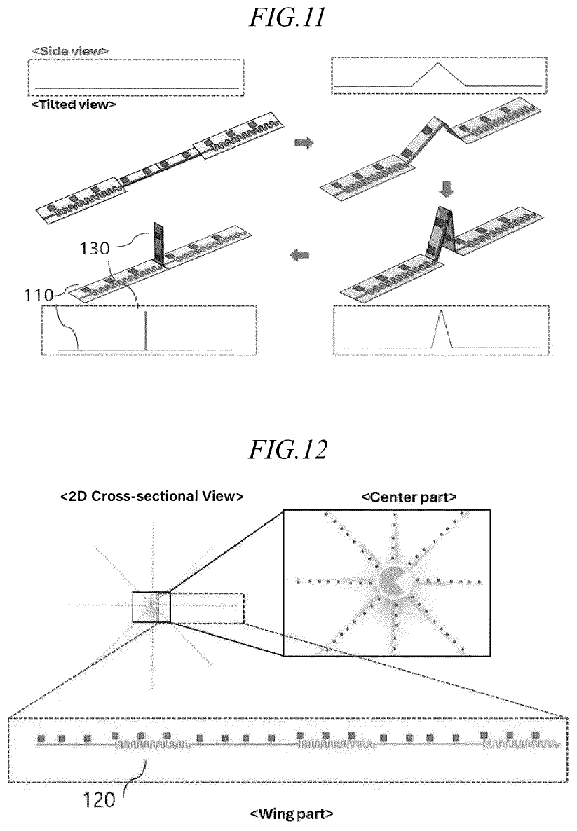

FIG. 12 is a plan view of a flexible three-dimensional electronic device having a wavy bending portion according to another embodiment of the present disclosure. Referring to FIG. 12, in the present embodiment, the plurality of flexible supports 110 may, respectively, include electrodes that have bending portions 120, each of which may take the form of waves with a bent structure or serpentine-shaped electrode. That is, in the present embodiment, the wavy form of electrode in the bent structure is formed on the flexible support, and thus the electrodes remain connected with each other without breaking when the flexible support bends.

### Embodiment 3

Yet another embodiment of the present disclosure provides a three-dimensional electronic device configured to conform to non-planar tissue surfaces using incision portions.

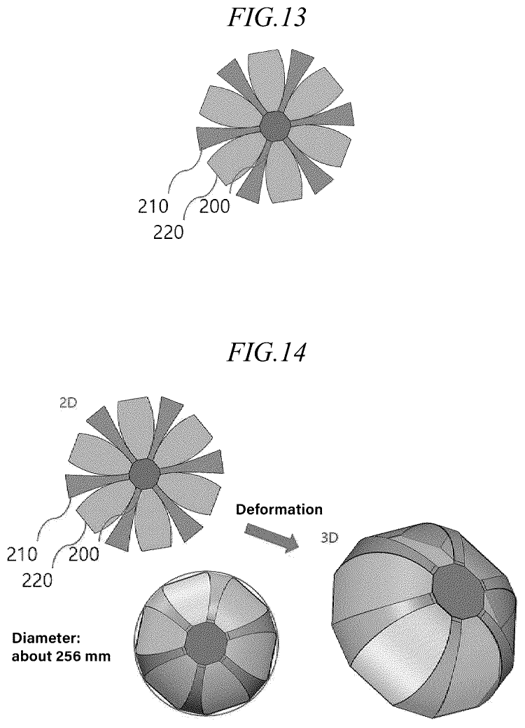

FIG. 13 is a plan view of a flexible three-dimensional electronic device for bio-conformal attachment according to yet another embodiment of the present disclosure. Referring to FIG. 13, the flexible three-dimensional electronic device may include a central support 200 having a predetermined area in a polygonal or circular shape, a plurality of first flexible supports 210 connected to the central support 200 and radially extending in multiple directions, and a plurality of second flexible supports 220 connected to the central support 200 and disposed between the first flexible supports 210.

Each of the plurality of first flexible supports 210 becomes wider toward its distal end from the central support 200, while each of the plurality of second flexible supports 220 becomes narrower toward its distal end. As a result, the gap between the first flexible support 210 and the second flexible support 220 (i.e., the width of the incision portion) is greatest at the distal ends of the first flexible support 210 and the second flexible support 220.

Additionally, the incision portion between the first flexible support and the second flexible support may be symmetrical with respect to the center, thereby enabling the device to envelop biological tissue from both sides.

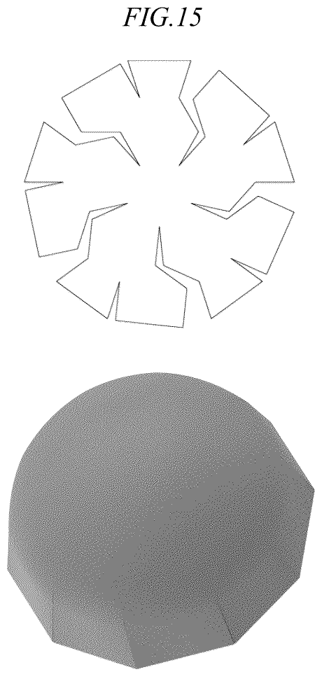

FIG. 14 illustrates how the flexible three-dimensional electronic device shown in FIG. 13 is used after attachment. Referring to FIG. 14, when the structure of FIG. 13 is applied, the device can be deformed into a substantially hemispherical shape with minimal gaps. Specifically, the gap area as a percentage of the total device area can be kept below 2%.

FIG. 15 is a plan view of a flexible three-dimensional electronic device according to yet another embodiment of the present disclosure. Referring to FIG. 15, in the present embodiment, the incision portions are not straight but instead formed in a zigzag pattern. When bending deformation is applied to this planar structure, the zigzag incision portions naturally close and join together to form a hemispherical shape.

### Embodiment 4

The electronic device according to the present disclosure includes, for adhesion to biological organs, a plurality of flexible supports 110 (see FIG. 1) extending radially in multiple directions from a central support, as described above. In this context, deformation of the flexible supports is critically important.

Accordingly, in the present embodiment, the deformation ratios of the flexible supports have been investigated through simulation to present appropriate design parameters.

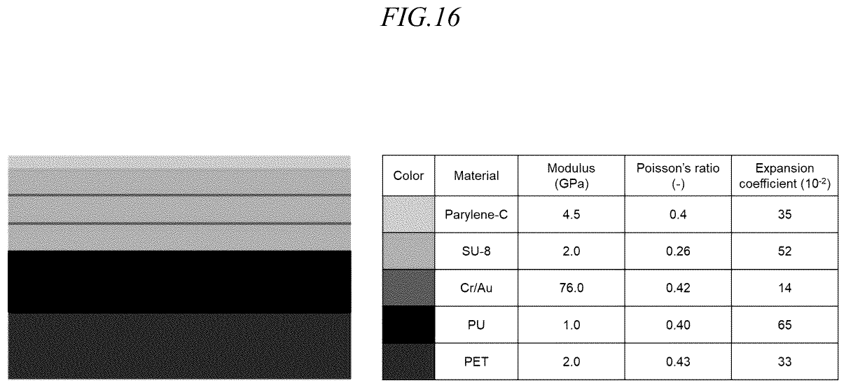

FIG. 16 is a cross-sectional view illustrating the interlayer structure of a flexible support according to the present embodiment, along with a table showing physical property parameters for each material. The test model has simulated an actual multilayer flexible support composed of layers having distinct physical variables (e.g., modulus, Poisson's ratio, and expansion coefficient). Particularly, electrodes are on a polyurethane substrate in the structure.

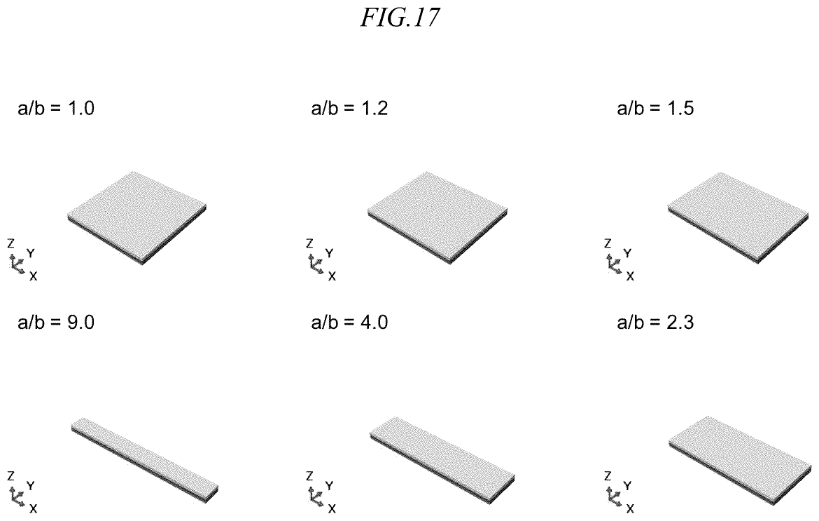

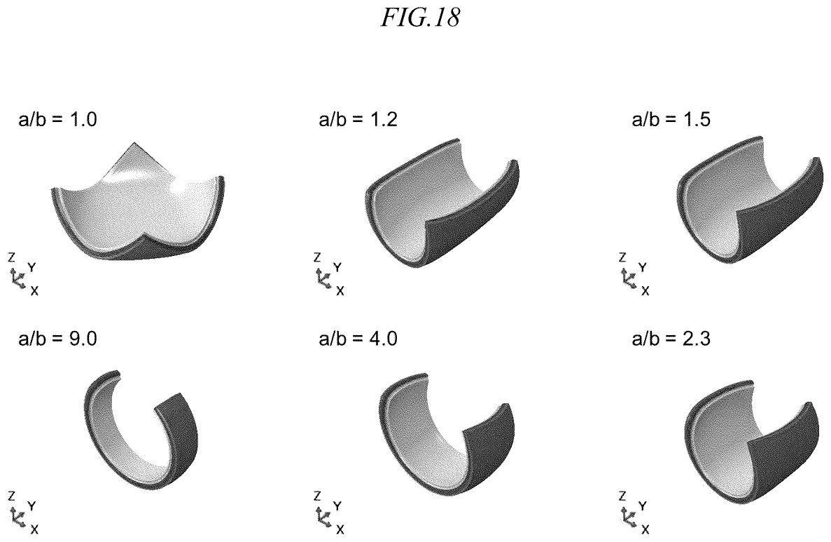

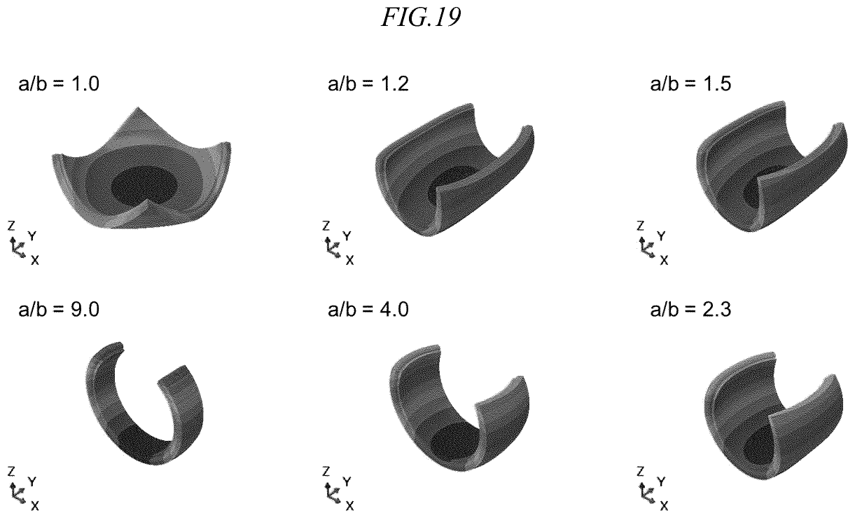

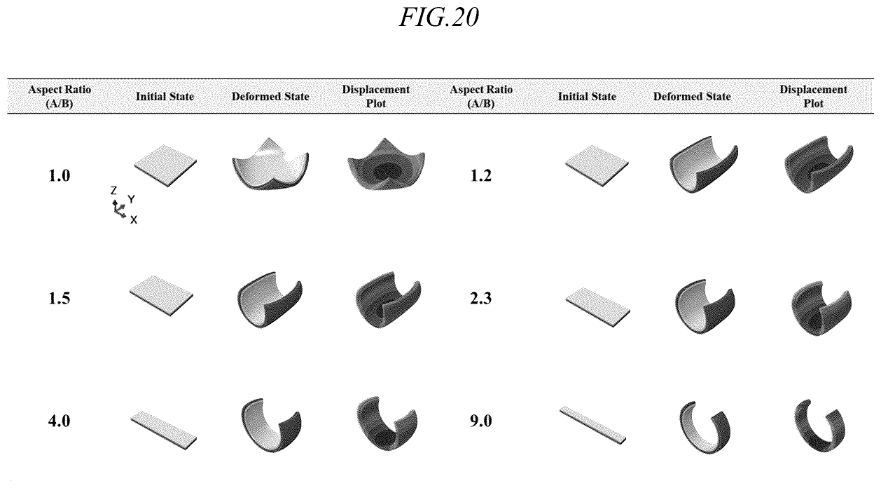

FIGS. 17 to 19 show, respectively, the initial state, deformed state, and displacement plot of a substrate forming the flexible support, tested using different aspect ratios of the substrate, and FIG. 20 shows the summarized results from these tests.

Referring to FIGS. 17 through 20, it is confirmed that when the aspect ratio is 4.0 or greater, deformation becomes significantly easier. As a result, substantial structural transformation of the device can be induced by environmental changes, enabling the device to autonomously adapt and adhere to the non-planar surfaces of target organs.

As described above, the flexible three-dimensional electronic device for bio-conformal attachment according to the present disclosure achieves full adhesion to target organs of various sizes and shapes and is capable of treating a wide range of internal organs by utilizing its structural adaptability. Furthermore, the device is capable of reducing the need for prolonged open surgery, enabling more efficient therapeutic procedures. In addition, the flexibility and three-dimensional conformal structure allow for tight contact with curved surfaces, facilitating effective applications such as phototherapy.

What is claimed is:

1. A flexible three-dimensional electronic device for bio-conformal attachment, comprising:
a plurality of light sources; and
a support having the plurality of light sources attached to one surface thereof,
wherein the support comprises a plurality of incision portions, and the incision portions become wider outward from a center.

2. The flexible three-dimensional electronic device of claim 1, wherein the incision portions are symmetrical about the support.

3. A flexible three-dimensional electronic device for bio-conformal attachment, comprising:
a central support having a predetermined area;
a plurality of flexible supports connected to the central support and radially extending in multiple directions; and
a plurality of light sources attached, respectively, to the plurality of flexible supports.

4. The flexible three-dimensional electronic device for bio-conformal attachment of claim 3, wherein the plurality of flexible supports comprise bending portions.

5. The flexible three-dimensional electronic device for bio-conformal attachment of claim 4, wherein the plurality of flexible supports are configured to be bent autonomously in response to internal physiological conditions.

6. The flexible three-dimensional electronic device for bio-conformal attachment of claim 3, wherein the flexible supports are provided with a flexible insertion member attached to the central support and configured to be inserted toward biological tissue to which the flexible three-dimensional electronic device is adhered.

7. The flexible three-dimensional electronic device for bio-conformal attachment of claim 6, wherein the flexible insertion member is a protruding structure formed by folding a portion of the flexible support.

8. The flexible three-dimensional electronic device for bio-conformal attachment of claim 7, wherein the flexible insertion member is provided with a plurality of light sources.

9. The flexible three-dimensional electronic device for bio-conformal attachment of claim 3, wherein an aspect ratio of the plurality of flexible supports is equal to or greater than 4.0.

10. A flexible three-dimensional electronic device for bio-conformal attachment, comprising:
a central support in a polygonal or circular shape to which a plurality of light sources is attached and having a predetermined area; and
a plurality of flexible supports connected to the central support and radially extending in multiple directions,
wherein incision portions between the flexible supports are symmetrical about the central support.

11. The flexible three-dimensional electronic device for bio-conformal attachment of claim 10, wherein the flexible supports comprise second flexible supports disposed between first flexible supports,
wherein the first flexible supports become wider toward distal ends thereof from the central support, and the second flexible supports become narrower toward distal ends thereof from the central support, such that gaps between the first flexible supports and the second flexible supports are greatest at their respective distal ends.

12. The flexible three-dimensional electronic device for bio-conformal attachment of claim 11, wherein the first flexible supports and the second flexible supports are configured such that, when the device is attached to biological tissue and bends in one direction, the distal ends of the first and second flexible supports meet each other.

13. The flexible three-dimensional electronic device for bio-conformal attachment of claim 11, wherein the distal ends of the first flexible supports and the second flexible supports meet to form a hemispherical shape and thus enable the device to conform to curvatures of organs and tissues.
